(19) United States
(12) Patent Application Publication
(10) Pub. No.: US 2010/0305154 A1
(43) Pub. Date: Dec. 2, 2010

(54) Prolyl Hydroxylase Inhibitors

(75) Inventor: Duke M. Fitch, King of Prussia, PA (US)

Correspondence Address: GlaxoSmithKline;GLOBAL PATENTS -US, UW2220, P. O. BOX 1539, KING OF PRUSSIA, PA, 19406-0939, US

(73) Assignee: GlaxoSmithKline LLC

(21) Appl. No.: 12/744,706

(22) PCT Filed: Nov. 26, 2008
(86) PCT No.: PCT/US08/84799

§ 371 Date: May 26, 2010

Related U.S. Application Data
(60) Provisional application No. 60/991,283, filed on Nov. 30, 2007.

(51) Int. Cl. A61K 31/47; C07D 215/04; A61P 7/06

(52) U.S. Cl. 514/311; 546/169

(57) ABSTRACT

The invention described herein relates to certain quinoline-8-carboxamide derivatives of formula (I)

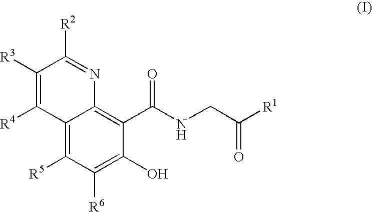

which are antagonists of HIF prolyl hydroxylases and are useful for treating diseases benefiting from the inhibition of this enzyme, anemia being one example.

### FIELD OF THE INVENTION

This invention relates to certain quinoline-8-carboxamide derivatives that are inhibitors of HIF prolyl hydroxylases, and thus have use in treating diseases benefiting from the inhibition of this enzyme, anemia being one example.

### BACKGROUND OF THE INVENTION

Anemia occurs when there is a decrease or abnormality in red blood cells, which leads to reduced oxygen levels in the blood. Anemia occurs often in cancer patients, particularly those receiving chemotherapy. Anemia is often seen in the elderly population, patients with renal disease, and in a wide variety of conditions associated with chronic disease.

Frequently, the cause of anemia is reduced erythropoietin (Epo) production resulting in prevention of erythropoiesis (maturation of red blood cells). Epo production can be increased by inhibition of prolyl hydroxylases that regulate hypoxia inducible factor (HIF).

One strategy to increase erythropoietin (Epo) production is to stabilize and thus increase the transcriptional activity of the HIF. HIF-alpha subunits (HIF-1 alpha, HIF-2αlpha, and HIF-3alpha) are rapidly degraded by proteosome under normoxic conditions upon hydroxylation of proline residues by prolyl hydroxylases (EGLN1, 2, 3). Proline hydroxylation allows interaction with the von Hippel Lindau (VHL) protein, a component of an E3 ubiquitin ligase. This leads to ubiquitination of HIF-alpha and subsequent degradation. Under hypoxic conditions, the inhibitory activity of the prolyl hydroxylases is suppressed, HIF-alpha subunits are therefore stabilized, and HIF-responsive genes, including Epo, are transcribed. Thus, inhibition of prolyl hydroxylases results in increased levels of HIF-alpha and thus increased Epo production.

The compounds of this invention provide a means for inhibiting these hydroxylases, increasing Epo production, and thereby treating anemia. Ischemia, stroke, and cytoprotection may also benefit by administering these compounds.

### SUMMARY OF THE INVENTION

In the first instance, this invention relates to a compound of formula (I):

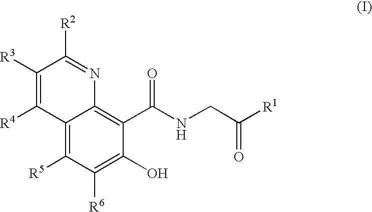

wherein:

R¹ is —NR⁷R⁸ or —OR⁹;

R², R³, R⁴, R⁵, and R⁶ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CONR¹⁰R¹¹, —N(R¹⁰)C(O)R¹², —N(R¹⁰)C(O)OR¹², —OC(O)NR¹⁰R¹¹, —P(O)(OR¹²)₂, SO₂NR¹⁰R¹¹—N(R¹⁰)SO₂R¹², C₁₀ alkyl, C₁-C₁₀ alkenyl, C₁-C₁₀ alkynyl, C₃-C₈ cycloalkyl, C₃-C₈ heterocycloalkyl, C₅-C₈ cycloalkenyl, aryl, and heteroaryl;

R⁷ and R⁸ are each independently selected from the group consisting of hydrogen, C₁-C₆ alkyl, C₂-C₆ alkenyl, C₂-C₆ alkynyl, C₃-C₆ cycloalkyl, C₃-C₆ heterocycloalkyl, aryl, and heteroaryl;

R⁹ is hydrogen, or a cation, or C₁-C₄ alkyl;

R¹⁰ and R¹¹ are each independently selected from the group consisting of hydrogen, C₁-C₁₀ alkyl, C₃-C₈ cycloalkyl, alkyl-C₃-C₈ cycloalkyl, C₃-C₈ heterocycloalkyl, C₁-C₁₀ alkyl-C₃-C₈ heterocycloalkyl, aryl, C₁-C₁₀ alkyl-aryl, heteroaryl, C₁-C₁₀ alkyl-heteroaryl, —CO(C₁-C₄—CO(C₃-C₆ cycloalkyl), —CO(C₃-C₆ heterocycloalkyl), —CO(aryl), —CO(heteroaryl), and —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;

each R¹² is independently selected from the group consisting of hydrogen, C₁-C₁₀ alkyl, C₂-C₁₀ alkenyl, C₂-C₁₀ alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈ heterocycloalkyl, aryl, C₁-C₁₀ alkyl-aryl, heteroaryl, and C₁-C₁₀ alkyl-heteroaryl;

any carbon or heteroatom of R², R³, R⁴, R⁵, R⁶, R⁷, R₈, R⁹, R¹⁰, R¹¹ or R¹², is unsubstituted or, where possible, is substituted with one or more substituents independently selected from C₁-C₆ alkyl, aryl, heteroaryl, halogen, —OR¹², —NR¹⁰R¹¹, cyano, nitro, —C(O)R¹², —C(O)OR¹², —SR¹², —S(O)R¹², —S(O)₂R¹², —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, N(R¹⁰)SO₂R¹², C₁-C₁₀ alkenyl, C₁-C₁₀ alkynyl, C₃-C₈ cycloalkyl, C₃-C₈ heterocycloalkyl, C₅-C₈ cycloalkenyl, aryl or heteroaryl, wherein R¹⁰, R¹¹, and R¹² are the same as defined above;

or a pharmaceutically acceptable salt or solvate thereof.

In a second aspect of the present invention, there is provided a compound of formula (I) or a salt or solvate thereof for use in mammalian therapy, e.g. treating amenia. An example of this therapeutic approach is that of a method for treating anemia caused by increasing the production of erythropoietin (Epo) by inhibiting HIF prolyl hydroxylases comprising administering a compound of formula (I) to a patient in need thereof, neat or admixed with a pharmaceutically acceptable excipient, in an amount sufficient to increase production of Epo.

In a third aspect of the present invention, there is provided a pharmaceutical composition comprising a compound of formula (I) or a salt, solvate, or the like thereof, and one or more of pharmaceutically acceptable carriers, diluents and excipients.

In a fourth aspect, there is provided the use of a compound of formula (I) or a salt or solvate thereof in the preparation of a medicament for use in the treatment of a disorder mediated by inhibiting HIF prolyl hydroxylases, such as an anemia, that can be treated by inhibiting HIF prolyl hydroxylases.

### DETAILED DESCRIPTION OF THE INVENTION

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁-C₄ alkyl” and “C₁-C₁₀ alkyl” refers to an alkyl group having at least 1 and up to 4 or 10 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, n-octyl, n-nonyl, and n-decyl, and branched analogs of the latter 5 normal alkanes.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “C₃-C₈ cycloalkyl” refers to a non-aromatic cyclic hydrocarbon ring having from three to eight carbon atoms. Exemplary “C₃-C₈ cycloalkyl” groups useful in the present invention include, but are not limited to, cyclopropyl, cyclobutyl, cyclopentyl, cyclohexyl, cycloheptyl and cyclooctyl.

The term “C₅-C₈ cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃-C₈ heterocycloalkyl” is used, it means a non-aromatic heterocyclic ring containing the specified number of ring atoms being, saturated or having one or more degrees of unsaturation and containing one or more heteroatom substitutions selected from O, S and/or N. Such a ring may be optionally fused to one or more other “heterocyclic” ring(s) or cycloalkyl ring(s). Examples of “heterocyclic” moieties include, but are not limited to, aziridine, thiirane, oxirane, azetidine, oxetane, thietane, tetrahydrofuran, pyran, 1,4-dioxane, 1,4-dithiane, 1,3-dioxane, 1,3-dioxolane, piperidine, piperazine, 2,4-piperazinedione, pyrrolidine, 2-imidazoline, imidazolidine, pyrazolidine, pyrazoline, morpholine, thiomorpholine, tetrahydrothiopyran, tetrahydrothiophene, and the like.

“Aryl” refers to optionally substituted monocyclic and polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl and the like.

“Heteroaryl” means an optionally substituted aromatic monocyclic ring or polycarbocyclic fused ring system wherein at least one ring complies with Hückel's Rule, has the specified number of ring atoms, and that ring contains at least one heteratom selected from N, O, and/or S. Examples of “heteroaryl” groups include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridinyl, pyridazinyl, pyrazinyl, pyrimidinyl, quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, benzofuranyl, benzothiophenyl, benzimidazolyl, benzthiazolyl, indolizinyl, indolyl, isoindolyl, and indazolyl.

The term “optionally” means that the subsequently described event(s) may or may not occur, and includes both event(s), which occur, and events that do not occur.

The term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group, one acidic enough to form salts. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically-acceptable acid addition salts by treatment with a suitable acid. Suitable acids include pharmaceutically-acceptable inorganic acids amd pharmaceutically-acceptable organic acids. Representative pharmaceutically-acceptable acid addition salts include hydrochloride, hydrobromide, nitrate, methylnitrate, sulfate, bisulfate, sulfamate, phosphate acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

Compounds of particular interest include those wherein:

R¹ is —NR⁷R⁸ or —OR⁹;

R², R³, R⁴, R⁵, and R⁶ are each independently selected from the group consisting of hydrogen, cyano, halogen, —C(O)R¹², —C(O)OR¹², OR¹², —NR¹⁰R¹¹, —CONR¹⁰R¹¹, —N(R¹⁰)C(O)R¹², —N(R¹⁰)C(O)N¹⁰, R¹¹, C₁₀ alkyl, C₁-C₁₀ alkenyl, C₁-C₁₀ alkynyl, C₃-C₈ cycloalkyl, C₃-C₈ heterocycloalkyl, C₅-C₈ cycloalkenyl, aryl, and heteroaryl;

R⁷ and R⁸ are each independently selected from the group consisting of hydrogen, C₁-C₆ alkyl, C₂-C₆ alkenyl, C₂-C₆ alkynyl, C₃-C₆ cycloalkyl, C₃-C₆ heterocycloalkyl, aryl, and heteroaryl;

R⁹ is hydrogen, or a cation, or C₁-C₄ alkyl;

R¹⁰ and R¹¹ are each independently selected from the group consisting of hydrogen, C₁-C₆ alkyl, C₃-C₆ cycloalkyl, C₃-C₆ heterocycloalkyl, aryl, heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;

each R¹² is independently selected from the group consisting of hydrogen, C₁-C₆ alkyl, C₂-C₆ alkenyl, C₂-C₆ alkynyl, —CO(C₁-C₄ alkyl), —CO(aryl), —CO(heteroaryl), —CO(C₃-C₆ cycloalkyl), —CO(C₃-C₆ heterocycloalkyl), C₃-C₆ cycloalkyl, C₃-C₆ heterocycloalkyl, aryl, and heteroaryl;

any carbon or heteroatom of R², R³, R⁴, R⁵, R⁶, R⁷, R⁸, R⁹, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, —C(O)R¹², —C(O)OR¹², —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰, R¹¹ and R¹² are the same as defined above;

Compounds of further interest are those wherein:

R¹ is —OR⁹;

R², R³, R⁴, R⁵, and R⁶ are each independently selected from the group consisting of hydrogen, cyano, halogen, —OR¹², —NR¹⁰R¹¹, C₁-C₆ alkyl, C₃-C₆ cycloalkyl, C₃-C₆ heterocycloalkyl, aryl, and heteroaryl;

R⁹ is hydrogen, or a cation;

R¹⁰ and R¹¹ are each independently selected from the group consisting of hydrogen, C₁-C₆ alkyl, C₃-C₆ cycloalkyl, C₃-C₆ heterocycloalkyl, aryl, heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;

each R¹² is independently selected from the group consisting of hydrogen, C₁-C₆ alkyl, C₂-C₆ alkenyl, C₂-C₆ alkynyl, —CO(C₁-C₄ alkyl), —CO(aryl), —CO(heteroaryl), —CO(C₃-C₆ cycloalkyl), —CO(C₃-C₆ heterocycloalkyl), C₃-C₆ cycloalkyl, C₃-C₆ heterocycloalkyl, aryl, and heteroaryl;

any carbon or heteroatom of R², R³, R⁴, R⁵, R⁶, R⁹, R¹⁰, R¹¹, or R¹² is unsubstituted or, where possible, is substituted with one or more substituents independently selected from C₁-C₆ alkyl, aryl, heteroaryl, halogen, —OR¹², —NR¹⁰R¹¹, cyano, —C(O)R¹², —C(O)OR¹², —CONR¹⁰R¹¹, N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰, R¹¹, and R¹² are the same as defined above;

Of further interest are those compounds where:

R¹ is —OR⁹;

R², R⁴, and R⁵ are each hydrogen;

R³ and R⁶ are each independently selected from the group consisting of hydrogen, cyano, halogen, —OR¹², NR¹⁰R¹¹CONR¹⁰R¹¹, C₁-C₆ alkyl, C₃-C₆ cycloalkyl, C₃-C₆ heterocycloalkyl, aryl, and heteroaryl;

R⁹ is hydrogen, or a cation;

R¹⁰ and R¹¹ are each independently selected from the group consisting of hydrogen, C₁-C₆ alkyl, C₃-C₆ cycloalkyl, C₃-C₆ heterocycloalkyl, aryl, and heteroaryl; or R¹⁰ and R¹¹ taken together with the nitrogen to which they are attached form a 5- or 6- or 7-membered saturated ring optionally containing one other heteroatom which is oxygen, nitrogen or sulphur;

each R¹² is independently selected from the group consisting of hydrogen, C₁-C₆ alkyl, C₃-C₆ cycloalkyl, C₃-C₆ heterocycloalkyl, aryl, and heteroaryl;

any carbon or heteroatom of R³, R⁶, R⁹, R¹⁰, R¹¹, or R¹² is unsubstituted or, where possible, is substituted with one or more substituents independently selected from C₁-C₆ alkyl, aryl, heteroaryl, halogen, —OR¹², —NR¹⁰R¹¹, cyano, —C(O)R¹², —C(O)OR¹², —CONR¹⁰, R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰, R¹¹, and R¹² are the same as defined above;

Specific compounds exemplified herein are:
1) N-[(7-hydroxy-8-quinolinyl)carbonyl]glycine;
2) N-[(7-hydroxy-3-phenyl-8-quinolinyl)carbonyl]glycine;
3) N-[(3-bromo-7-hydroxy-8-quinolinyl)carbonyl]glycine;
4) N-({3-[4-(1,1-dimethylethyl)phenyl]-7-hydroxy-8-quinolinyl}carbonyl)glycine;
5) N-({7-hydroxy-3-[4-(trifluoromethyl)phenyl]-8-quinolinyl}carbonyl)glycine;
6) N-[(3,6-dibromo-7-hydroxy-8-quinolinyl)carbonyl]glycine;
7) N-[(7-hydroxy-3,6-diphenyl-8-quinolinyl)carbonyl]glycine;
8) N-({3,6-bis[4-(1,1-dimethylethyl)phenyl]-7-hydroxy-8-quinolinyl}carbonyl)glycine; and
9) N-{[3,6-bis(3,5-difluorophenyl)-7-hydroxy-8-quinolinyl]carbonyl}glycine;

Processes for preparing the compound of formula (I) are also within the ambit of this invention. To illustrate, process for preparing a compound of formula (I)

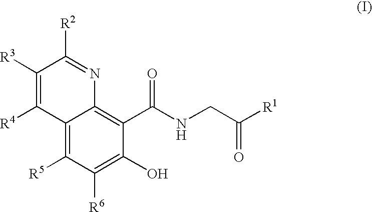

wherein R¹, R², R³, R⁴, R⁵, and R⁶ are the same as defined above for formula (I), the process comprising treating a compound of formula A:

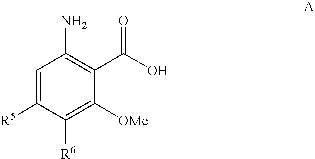

wherein R⁵ and R⁶ are the same as for those groups in formula (I), with an appropriately substituted α,β-unsaturated carbonyl compound, such as acrolein, 2-phenylpropenal, or 2-bromoacrolein, in an appropriate solvent, such as acetic acid or 1,4-dioxane, with or without the addition of bromine, with heating under either conventional thermal conditions or by microwave irradiation, to form a compound of formula B:

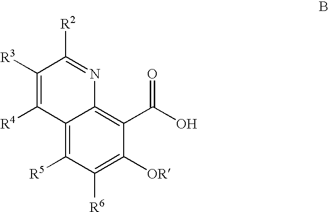

wherein R², R³, R⁴, R⁵, and R⁶ are the same as for those groups in formula (I) and R′ is H or Me, which is coupled with an appropriate glycine ester, such as glycine ethyl ester hydrochloride, and an appropriate base, such as triethylamine, and an appropriate coupling reagent, such as HATU, in an appropriate solvent, such as N,N-dimethylformamide, followed by ester hydrolysis with an appropriate base, such as sodium hydroxide, in an appropriate solvent, such as tetrahydrofuran and/or methanol, or when necessary, ether cleavage/ester hydrolysis with an appropriate reagent, such as boron tribromide, in an appropriate solvent, such as dichloromethane, to form a compound of formula (I) where R¹ is —OH.

The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain of the compounds described herein may contain one or more chiral atoms, or may otherwise be capable of existing as two enantiomers. The compounds claimed below include mixtures of enantiomers as well as purified enantiomers or enantiomerically enriched mixtures. Also included within the scope of the invention are the individual isomers of the compounds represented by formula (I), or claimed below, as well as any wholly or partially equilibrated mixtures thereof. The present invention also covers the individual isomers of the claimed compounds as mixtures with isomers thereof in which one or more chiral centers are inverted. Also, it is understood that any tautomers and mixtures of tautomers of the claimed compounds are included within the scope of the compounds of formula (I) as disclosed herein above or claimed herein below.

Where there are different isomeric forms they may be separated or resolved one from the other by conventional methods, or any given isomer may be obtained by conventional synthetic methods or by stereospecific or asymmetric syntheses.

While it is possible that, for use in therapy, a compound of formula (I), as well as salts, solvates and the like, may be administered as a neat preparation, i.e. no additional carrier, the more usual practice is to present the active ingredient confected with a carrier or diluent. Accordingly, the invention further provides pharmaceutical compositions, which includes a compound of formula (I) and salts, solvates and the like, and one or more pharmaceutically acceptable carriers, diluents, or excipients. The compounds of formula (I) and salts, solvates, etc, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates etc, with one or more pharmaceutically acceptable carriers, diluents or excipients.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavouring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of formula (I) for the treatment of anemia will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, etc., may be determined as a proportion of the effective amount of the compound of formula (I) per se. It is envisaged that similar dosages would be appropriate for treatment of the other conditions referred to above.

### DEFINITIONS

MgSO₄—Magnesium sulfate,
NH₄OH—Ammonium hydroxide,
HATU—2-(1H-7-azabenzotriazol-1-yl)-1,1,3,3-tetramethyluronium hexafluorophosphate.

### Chemical Background:

The compounds of this invention may be made by a variety of methods, including standard chemistry. Any previously defined variable will continue to have the previously defined meaning unless otherwise indicated. Illustrative general synthetic methods are set out below and then specific compounds of the invention as prepared are given in the examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognize if a stereocenter exists in compounds of formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

The compounds described herein may be made from commercially available starting materials or synthesized using known organic, inorganic and/or enzymatic processes.

### Illustrated Methods of Preparation

### Schemes

Included in the present invention is a process according to Scheme 1 for the synthesis of the compounds:

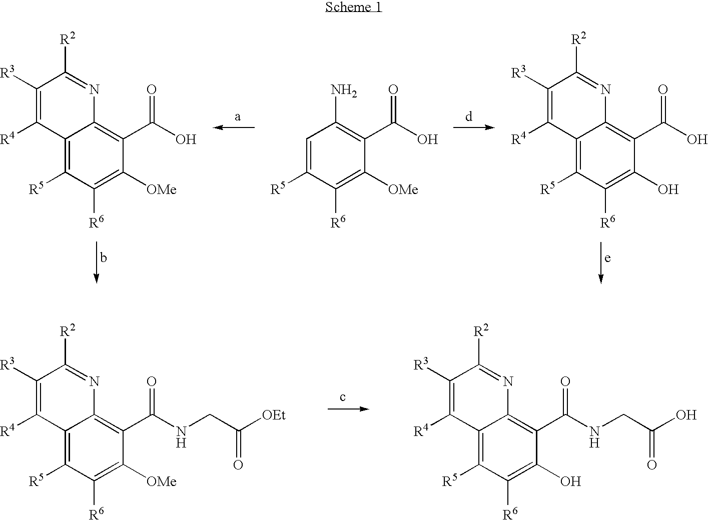

a) R⁴CH(R³)CC(O)R², 1,4-dioxane, μwave; b) glycine ethyl ester hydrochloride, triethylamine, HATU, N,N-dimethylformamide; c) boron tribromide, dichloromethane; d) R⁴CH(R³)CC(O)R², bromine, acetic acid or 1,4-dioxane, Δ; e) glycine ethyl ester hydrochloride, triethylamine, HATU, N,N-dimethylformamide then NaOH, tetrahydrofuran, methanol.

### Example 1

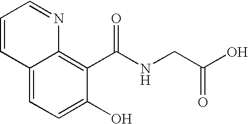

### N-[7-hydroxy-8-quinolinyl)carbonyl]glycine

### 1a) 7-(methyloxy)-8-quinolinecarboxylic acid

A mixture of 2-amino-6-(methyloxy)benzoic acid (1.00 g, 6.00 mmol) and acrolein (0.445 mL, 6.00 mmol) in 1,4-dioxane (6.0 mL) was heated to 200° C. for 20 min. in a Biotage Initiator® microwave synthesizer (http://www.biotage.com). The mixture was concentrated in vacuo and purified via flash column chromatography (0-10% methanol in dichloromethane) to afford the title compound (0.430 g, 35%) as a light orange solid. ¹H NMR (400 MHz, DMSO-d6) δ ppm 13.1 (br. s., 1H), 8.85 (dd, J=4.3, 1.8 Hz, 1H), 8.35 (dd, J=8.3, 1.8 Hz, 1H), 8.06 (d, J=9.1 Hz, 1H), 7.60 (d, J=9.1 Hz, 1H), 7.43 (dd, J=8.3, 4.3 Hz, 1H), 3.96 (s, 3H). MS (ES+) m/e 204 [M+H]+.

### 1b) Ethyl N-{[7-(methyloxy)-8-quinolinyl]carbonyl}glycinate

To a solution of the compound from Example 1a) (0.203 g, 1.00 mmol) and glycine ethyl ester hydrochloride (0.279 g, 2.00 mmol) in N,N-dimethylformamide (5.0 mL) were added triethylamine (0.418 mL, 3.00 mmol) and HATU (0.418 g, 1.10 mmol). The reaction mixture was stirred overnight at ambient temperature, quenched by water, diluted with brine, and extracted twice with ethyl acetate. The combined organic layers were dried over MgSO₄, filtered, concentrated in vacuo, and purified via flash column chromatography (0-10% methanol in ethyl acetate) to afford the title compound (0.236 g, 82%) as a light orange oil. ¹H NMR (400 MHz, DMSO-d6) δ ppm 8.83 (dd, J=4.3, 1.8 Hz, 1H), 8.55 (t, J=5.8 Hz, 1H), 8.32 (dd, J=8.1, 1.8 Hz, 1 H), 8.03 (d, J=9.1 Hz, 1H), 7.57 (d, J=9.1 Hz, 1H), 7.39 (dd, J=8.2, 4.2 Hz, 1H), 4.15 (q, J=7.1 Hz, 2H), 4.05 (d, J=5.8 Hz, 2H), 3.93 (s, 3H), 1.25 (t, J=7.1 Hz, 3H). MS (ES+) m/e 289 [M+H]+.

### 1c) N-[(7-hydroxy-8-quinolinyl)carbonyl]glycine

To a solution of the compound from Example 1b) (0.236 g, 0.819 mmol) in dichloromethane (2.0 mL) was added boron tribromide (1M solution in dichloromethane) (2.46 mL, 2.46 mmol). The reaction mixture was stirred 2 h at ambient temperature, quenched by water, diluted with brine, and extracted twice with ethyl acetate. The combined organic layers were dried over MgSO₄, filtered, concentrated in vacuo, and purified via flash column chromatography (0-10% methanol in dichloromethane followed by 1% NH₄OH and 10% methanol in dichloromethane) to afford the title compound (0.098 g, 49%) as a light yellow solid. ¹H NMR (400 MHz, DMSO-d6) δ ppm 15.8 (br. s., 1H), 12.0 (s, 1H), 8.88 (d, J=1.8 Hz, 1H), 8.42 (dd, J=8.1, 1.5 Hz, 1H), 8.06 (d, J=9.1 Hz, 1H), 7.49 (dd, J=7.6, 4.8 Hz, 1H), 7.25 (d, J=8.8 Hz, 1H), 3.89 (d, J=3.5 Hz, 1H). MS (ES+) m/e 247 [M+H]+.

### Example 2

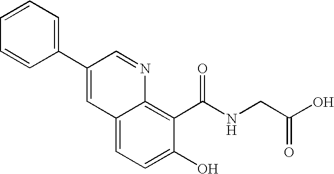

### N-[7-hydroxy-3-phenyl-8-quinolinyl)carbonyl]glycine

### 2a) 7-(methyloxy)-3-phenyl-8-quinolinecarboxylic acid

A mixture of 2-amino-6-(methyloxy)benzoic acid (0.250 g, 1.496 mmol) and 2-phenylpropenal (prepared by the method of Nsanzumuhire, C.; Clément, J.-L.; Ouari, O.; Karoui, H.; Finet, J.-P.; Tordo, P. Tetrahedron Lett. 2004, 45, 6385-6389) (0.198 g, 1.496 mmol) in 1,4-dioxane (2.0 mL) was heated to 200° C. for 20 min. in a Biotage Initiator® microwave synthesizer. The mixture was concentrated in vacuo and purified via flash column chromatography (0-10% methanol in dichloromethane) to afford the title compound (0.062 g, 15%) as a light orange solid. ¹H NMR (400 MHz, DMSO-d₆) δ ppm 13.1 (br. s., 1H), 9.22 (d, J=2.3 Hz, 1H), 8.64 (d, J=2.3 Hz, 1H), 8.13 (d, J=9.1 Hz, 1H), 7.87 (d, J=7.3 Hz, 2H), 7.64 (d, J=9.1 Hz, 1H), 7.55 (t, J=7.7 Hz, 2H), 7.45 (t, J=7.3 Hz, 1H), 3.98 (s, 3H). MS (ES+) m/e 280 [M+H]⁺.

### 2b) Ethyl N-{[7-(methyloxy)-3-phenyl-8-quinolinyl]carbonyl}glycinate

To a solution of the compound from Example 2a) (0.056 g, 0.201 mmol) and glycine ethyl ester hydrochloride (0.056 g, 0.401 mmol) in N,N-dimethylformamide (2.0 mL) were added triethylamine (0.084 mL, 0.602 mmol) and HATU (0.084 g, 0.221 mmol). The reaction mixture was stirred overnight at ambient temperature, quenched by water, diluted with brine, and extracted twice with ethyl acetate. The combined organic layers were dried over MgSO₄, filtered, concentrated in vacuo, and purified via flash column chromatography (20-100% ethyl acetate in hexanes) to afford the title compound (0.067 g, 92%) as a light orange oil. ¹H NMR (400 MHz, DMSO-d6) δ ppm 9.20 (d, J=2.5 Hz, 1H), 8.62 (d, J=2.5 Hz, 1H), 8.61 (t, J=5.8 Hz, 1H), 8.11 (d, J=9.1 Hz, 1H), 7.86 (d, J=7.3 Hz, 2H), 7.62 (d, J=9.1 Hz, 1H), 7.55 (t, J=7.6 Hz, 2H), 7.44 (t, J=7.3 Hz, 1H), 4.16 (q, J=7.2 Hz, 2H), 4.07 (d, J=5.8 Hz, 2H), 3.95 (s, 3H), 1.26 (t, J=7.2 Hz, 3H). MS (ES+) m/e 365 [M+H]+.

### 2c) N-[(7-hydroxy-3-phenyl-8-quinolinyl)carbonyl]glycine

To a solution of the compound from Example 2b) (0.062 g, 0.170 mmol) in dichloromethane (2.0 mL) was added boron tribromide (1M solution in dichloromethane) (0.510 mL, 0.510 mmol). The reaction mixture was stirred 5 h at ambient temperature, quenched by water, diluted with brine, and extracted twice with ethyl acetate. The combined organic layers were dried over MgSO₄, filtered, concentrated in vacuo, and purified via flash column chromatography (20-100% ethyl acetate in hexanes then 0-10% methanol in ethyl acetate) to afford the title compound (0.013 g, 24%) as a light yellow solid. ¹H NMR (400 MHz, DMSO-d₆) δ ppm 15.4 (s, 1H), 12.9 (br. s., 1H), 12.1 (t, J=5.4 Hz, 1H), 9.25 (d, J=2.5 Hz, 1H), 8.78 (d, J=2.5 Hz, 1H), 8.18 (d, J=9.1 Hz, 1H), 7.89 (d, J=7.1 Hz, 2H), 7.57 (t, J=7.6 Hz, 2H), 7.47 (t, J=7.5 Hz, 1H), 7.35 (d, J=9.1 Hz, 1H), 4.24 (d, J=5.6 Hz, 2H). MS (ES+) m/e 323 [M+H]⁺.

### Example 3

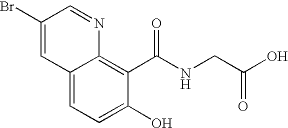

### N-[(3-bromo-7-hydroxy-8-quinolinyl)carbonyl]glycine

### 3a) 3-bromo-7-hydroxy-8-quinolinecarboxylic acid

A solution of 2-bromoacrolein (prepared by the method of Nicolaou, K. C.; Brenzovich, W. E.; Bulger, P. G.; Francis, T. M., Org. Biomol. Chem. 2006, 4, 2119-2157) (0.60 mL, 7.42 mmol) in glacial acetic acid (20.0 mL) at ambient temperature was titrated to the appearance of a faint reddish color with bromine (0.382 mL, 7.42 mmol). 2-amino-6-(methyloxy)benzoic acid (1.24 g, 7.42 mmol) was added, and the solution was heated to 100° C. for 2 h. Upon cooling, the solution was concentrated in vacuo and purified via flash column chromatography (20-100% ethyl acetate in hexanes) to afford the title compound (0.312 g, 16%) as a light yellow solid. ¹H NMR (400 MHz, DMSO-d₆) δ ppm 14.6 (s, 1H), 9.12 (d, J=2.3 Hz, 1H), 9.03 (d, J=2.3 Hz, 1H), 8.18 (d, J=9.3 Hz, 1H), 7.46 (d, J=9.3 Hz, 1H). MS (ES+) m/e 268/270 [M+H]+.

### 3b) Ethyl N-{[7-(methyloxy)-3-phenyl-8-quinolinyl]carbonyl}glycinate

To a solution of the compound from Example 3a) (0.150 g, 0.560 mmol) and glycine ethyl ester hydrochloride (0.312 g, 2.238 mmol) in N,N-dimethylformamide (5.0 mL) were added triethylamine (0.468 mL, 3.36 mmol) and HATU (0.468 g, 1.231 mmol). The reaction mixture was stirred overnight at ambient temperature, quenched by water, diluted with brine, and extracted twice with ethyl acetate. The combined organic layers were dried over MgSO₄, filtered, concentrated in vacuo, and purified via flash column chromatography (10-30% ethyl acetate in hexanes) to afford the title compound (0.110 g, 56%) as a white solid. ¹H NMR (400 MHz, DMSO-d₆) δ ppm 15.3 (s, 1H), 11.7 (t, J=5.6 Hz, 1H), 8.95 (d, J=2.5 Hz, 1H), 8.80 (d, J=2.5 Hz, 1H), 8.08 (d, J=9.1 Hz, 1H), 7.36 (d, J=9.1 Hz, 1H), 4.30 (d, J=5.6 Hz, 2H), 4.17 (q, J=7.1 Hz, 2H), 1.23 (t, J=7.1 Hz, 3H). MS (ES+) m/e 353/355 [M+H]⁺.

### 3c) N-[(3-bromo-7-hydroxy-8-quinolinyl)carbonyl]glycine

To a solution of the compound from Example 3b) (0.108 g, 0.306 mmol) in tetrahydrofuran (1.0 mL) and methanol (1.0 mL) was added 1N aqueous sodium hydroxide (1.00 mL, 1.00 mmol). After stirring 15 min. at ambient temperature, the reaction was quenched with 1N aqueous hydrochloric acid, diluted with brine, and extracted twice with ethyl acetate. The combined organic layers were concentrated in vacuo and triturated with 50% ethyl acetate in hexanes to afford the title compound (0.090 g, 91%) as an off-white solid. ¹H NMR (400 MHz, DMSO-d₆) δ ppm 15.4 (s, 1H), 12.9 (br. s., 1H), 11.7 (t, J=5.6 Hz, 1H), 8.96 (d, J=2.5 Hz, 1H), 8.80 (d, J=2.5 Hz, 1H), 8.08 (d, J=9.1 Hz, 1H), 7.36 (d, J=9.1 Hz, 1H), 4.22 (d, J=5.6 Hz, 2H). MS (ES+) m/e 325/327 [M+H]⁺.

### Example 4

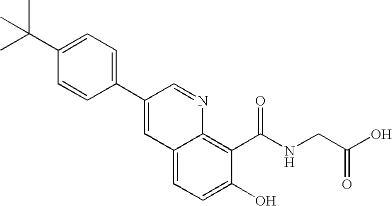

### N-({3-[4-(1,1-dimethylethyl)phenyl]-7-hydroxy-8-quinolinyl}carbonyl)glycine

A solution of the compound from Example 3c) (0.040 g, 0.123 mmol), (4-t-butylphenyl)boronic acid (0.033 g, 0.185 mmol), potassium carbonate (0.051 g, 0.369 mmol), and tetrakis(triphenylphosphine)palladium(0) (0.014 g, 0.012 mmol) in 1,4-dioxane (2.0 mL) was heated to 200° C. for 40 min. in a Biotage Initiator® microwave synthesizer. Upon cooling, the reaction mixture was treated with 1M aqueous hydrochloric acid, diluted with brine, and extracted twice with ethyl acetate. The combined organic layers were dried over MgSO₄, filtered, concentrated in vacuo, and purified via flash column chromatography (0-10% methanol in dichloromethane) to afford the title compound (0.038 g, 82%) as a yellow solid. ¹H NMR (400 MHz, DMSO-d₆) δ ppm 15.3 (s, 1H), 12.9 (br. s., 1H), 12.1 (t, J=5.6 Hz, 1H), 9.24 (d, J=2.5 Hz, 1H), 8.74 (d, J=2.3 Hz, 1H), 8.17 (d, J=9.1 Hz, 1H), 7.82 (d, J=8.6 Hz, 2H), 7.58 (d, J=8.6 Hz, 2H), 7.34 (d, J=8.8 Hz, 1H), 4.26 (d, J=5.6 Hz, 2H), 1.34 (s, 9H). MS (ES+) m/e 379 [M+H]⁺.

### Example 5

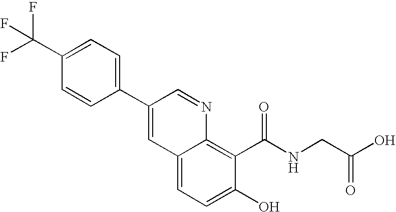

### N-({7-hydroxy-3-[4-(trifluoromethyl)phenyl]-8-quinolinyl}1 carbonyl)glycine

A solution of N-[(3-bromo-7-hydroxy-8-quinolinyl)carbonyl]glycine (prepared as in Example 3c) (0.040 g, 0.123 mmol), [4-(trifluoromethyl)phenyl]boronic acid (0.035 g, 0.185 mmol), potassium carbonate (0.051 g, 0.369 mmol), and tetrakis(triphenylphosphine)palladium(0) (0.014 g, 0.012 mmol) in 1,4-dioxane (2.0 mL) was heated to 200° C. for 40 min. in a Biotage Initiator® microwave synthesizer. Upon cooling, the reaction mixture was treated with 1M aqueous hydrochloric acid, diluted with brine, and extracted twice with ethyl acetate. The combined organic layers were dried over MgSO₄, filtered, concentrated in vacuo, and purified via flash column chromatography (0-10% methanol in dichloromethane) to afford the title compound (0.037 g, 77%) as a yellow solid. ¹H NMR (400 MHz, DMSO-d₆) δ ppm 15.4 (s, 1H), 12.9 (br. s., 1H), 12.1 (t, J=5.6 Hz, 1H), 9.31 (d, J=2.5 Hz, 1H), 8.89 (d, J=2.5 Hz, 1H), 8.20 (d, J=9.1 Hz, 1H), 8.14 (d, J=8.3 Hz, 2H), 7.92 (d, J=8.3 Hz, 2H), 7.38 (d, J=9.1 Hz, 1H), 4.26 (d, J=5.6 Hz, 2H). MS (ES+) m/e 391 [M+H]⁺.

### Example 6

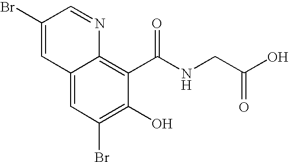

### N-[(3,6-dibromo-7-hydroxy-8-quinolinyl)carbonyl]glycine

To a suspension of N-[(3-bromo-7-hydroxy-8-quinolinyl)carbonyl]glycine (prepared as in Example 3c) (0.060 g, 0.185 mmol) in glacial acetic acid (2.0 mL) at ambient temperature was added bromine (0.029 mL, 0.554 mmol). Following stirring overnight at ambient temperature, the reaction mixture was diluted with hexanes, filtered, and washed with hexanes to afford the title compound (0.063 g, 84%) as a pale yellow solid. ¹H NMR (400 MHz, DMSO-d₆) δ ppm 12.96 (br. s., 1H), 11.78 (t, J=5.6 Hz, 1H), 8.99 (d, J=2.5 Hz, 1H), 8.77 (d, J=2.5 Hz, 1H), 8.58 (s, 1H), 4.24 (d, J=5.6 Hz, 2H). MS (ES+) m/e 405 [M+H]⁺.

### Example 7

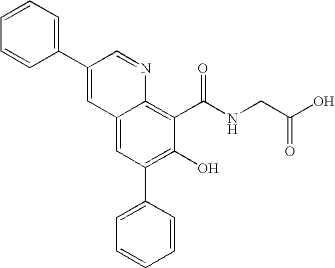

### N-[(7-hydroxy-3,6-diphenyl-8-quinolinyl)carbonyl]glycine

### 7a) 3,6-dibromo-7-hydroxy-8-quinolinecarboxylic acid

To a solution of 2-bromoacrolein (prepared by the method of Nicolaou, K. C.; Brenzovich, W. E.; Bulger, P. G.; Francis, T. M. Org. Biomol. Chem. 2006, 4, 2119-2157) (0.400 mL, 4.95 mmol) in 1,4-dioxane (20 mL) at ambient temperature was added bromine (0.270 mL, 5.24 mmol). Following stirring 10 min. at ambient temperature, 2-amino-6-(methyloxy)benzoic acid (0.800 g, 4.79 mmol) was added, and the solution was heated to reflux for 1 h. Upon cooling, the solution was concentrated in vacuo and purified via flash column chromatography (20-100% ethyl acetate in hexanes) to afford the title compound (0.720 g, 43%) as a yellow solid. ¹H NMR (400 MHz, DMSO-d₆) δ ppm 16.0 (br. s., 1H), 9.06 (d, J=2.3 Hz, 1H), 9.02 (d, J=2.3 Hz, 1H), 8.60 (s, 1H). MS (ES+) m/e 348 [M+H]⁺.

### 7b) Ethyl N-[(3,6-dibromo-7-hydroxy-8-quinolinyl)carbonyl]glycinate

To a solution of the compound from Example 7a) (0.280 g, 0.807 mmol) and glycine ethyl ester hydrochloride (0.451 g, 3.23 mmol) in N,N-dimethylformamide (5.0 mL) were added triethylamine (0.675 mL, 4.84 mmol) and HATU (0.675 g, 1.775 mmol). The reaction mixture was stirred overnight at ambient temperature, quenched by water, diluted with brine, and extracted twice with ethyl acetate. The combined organic layers were dried over MgSO₄, filtered, concentrated in vacuo, and purified via flash column chromatography (10-30% ethyl acetate in hexanes) to afford the title compound (0.307 g, 88%) as a light yellow solid. ¹H NMR (400 MHz, DMSO-d₆) δ ppm 11.8 (t, J=5.6 Hz, 1H), 8.98 (d, J=2.3 Hz, 1H), 8.77 (d, J=2.3 Hz, 1H), 8.57 (s, 1H), 4.32 (d, J=5.6 Hz, 2H), 4.17 (q, J=7.1 Hz, 2H), 1.23 (t, J=7.1 Hz, 3H). MS (ES+) m/e 433 [M+H]⁺.

### 7c) Ethyl N-[(7-hydroxy-3,6-diphenyl-8-quinolinyl)carbonyl]glycinate

A solution the compound from Example 7b) (0.100 g, 0.231 mmol), phenylboronic acid (0.056 g, 0.463 mmol), potassium carbonate (0.096 g, 0.694 mmol), and tetrakis(triphenylphosphine)palladium(0) (0.008 g, 0.0069 mmol) in 1,4-dioxane (1.5 mL) and water (0.5 mL) was heated to 100° C. for 20 min in a Biotage Initiator® microwave synthesizer. Upon cooling, the reaction mixture was treated with water, diluted with brine, and extracted twice with ethyl acetate. The combined organic layers were dried over MgSO₄, filtered, concentrated in vacuo, and purified via flash column chromatography (10-30% ethyl acetate in hexanes) to afford the title compound (0.091 g, 92%) as a white solid. ¹H NMR (400 MHz, DMSO-d₆) δ ppm 16.0 (s, 1H), 12.4 (t, J=5.7 Hz, 1H), 9.26 (d, J=2.5 Hz, 1H), 8.82 (d, J=2.5 Hz, 1H), 8.25 (s, 1H), 7.89 (d, J=7.1 Hz, 2H), 7.67 (d, J=7.1 Hz, 2H), 7.58 (t, J=7.6 Hz, 2H), 7.51 (t, J=7.1 Hz, 2H), 7.44-7.49 (m, 2H), 4.37 (d, J=5.7 Hz, 2H), 4.20 (q, J=7.1 Hz, 2H), 1.25 (t, J=7.1 Hz, 3H). MS (ES+) m/e 427 [M+H]⁺.

### 7d) N-[(7-hydroxy-3,6-diphenyl-8-quinolinyl)carbonyl]glycine

To a solution of the compound from Example 7c) (0.091 g, 0.213 mmol) in tetrahydrofuran (1.0 mL) and methanol (1.0 mL) was added 1N aqueous sodium hydroxide (1.0 mL, 1.00 mmol). After stirring 15 min. at ambient temperature, the reaction was quenched with 1N aqueous hydrochloric acid, diluted with brine, and extracted twice with ethyl acetate. The combined organic layers were concentrated in vacuo, washed with hexanes, and filtered to afford the title compound (0.082 g, 96%) as a yellow solid. ¹H NMR (400 MHz, DMSO-d₆) δ ppm 16.1 (s, 1H), 12.9 (br. s., 1H), 12.3 (t, J=5.6 Hz, 1H), 9.26 (d, J=2.3 Hz, 1H), 8.81 (d, J=2.3 Hz, 1H), 8.24 (s, 1H), 7.89 (d, J=7.3 Hz, 2H), 7.68 (d, J=6.8 Hz, 2H), 7.58 (t, J=7.6 Hz, 2H), 7.51 (t, J=7.1 Hz, 2H), 7.42-7.48 (m, 2H), 4.29 (d, J=5.6 Hz, 2H). MS (ES+) m/e 399 [M+H]⁺.

### Example 8

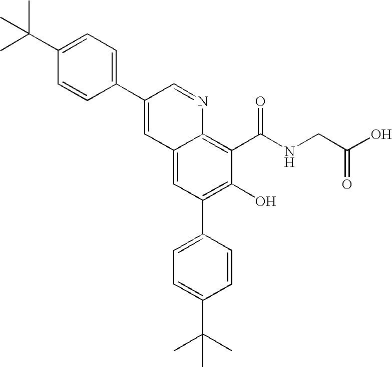

### N-({3,6-bis[4-(1,1-dimethylethyl)phenyl]-7-hydroxy-8-quinolinyl}carbonyl)glycine

### 8a) Ethyl N-({3,6-bis[4-(1,1-dimethylethyl)phenyl]-7-hydroxy-8-quinolinyl}carbonyl)glycinate

A solution of the compound from Example 7b) (0.050 g, 0.116 mmol), (4-t-butylphenyl)boronic acid (0.021 g, 0.116 mmol), potassium carbonate (0.048 g, 0.347 mmol), and tetrakis(triphenylphosphine)palladium(0) (0.004 g, 0.0035 mmol) in 1,4-dioxane (1.5 mL) and water (0.5 mL) was heated to 100° C. for 20 min in a Biotage Initiator® microwave synthesizer. Upon cooling, the reaction mixture was concentrated in vacuo and purified via flash column chromatography (0-30% ethyl acetate in hexanes) to afford the title compound (0.054 g, 87%) as a yellow solid. ¹H NMR (400 MHz, DMSO-d₆) δ ppm 16.0 (s, 1H), 12.4 (t, J=5.6 Hz, 1H), 9.24 (d, J=2.3 Hz, 1H), 8.75 (d, J=2.3 Hz, 1H), 8.22 (s, 1H), 7.81 (d, J=8.6 Hz, 2H), 7.61 (d, J=8.3 Hz, 2H), 7.59 (d, J=8.6 Hz, 2H), 7.52 (d, J=8.6 Hz, 2H), 4.37 (d, J=5.6 Hz, 2H), 4.19 (q, J=7.1 Hz, 2H), 1.35 (s, 9H), 1.35 (s, 9H), 1.25 (t, J=7.1 Hz, 3H). MS (ES+) m/e 539 [M+H]⁺.

### 8b) N-({3,6-bis[4-(1,1-dimethylethyl)phenyl]-7-hydroxy-8-quinolinyl}carbonyl)glycine

To a solution of the compound from Example 8a) (0.054 g, 0.100 mmol) in tetrahydrofuran (1.0 mL) and methanol (1.0 mL) was added 1N aqueous sodium hydroxide (1.0 mL, 1.00 mmol). After stirring 15 min. at ambient temperature, the reaction was quenched with 1N aqueous hydrochloric acid, diluted with brine, and extracted twice with ethyl acetate. The combined organic layers were dried over MgSO₄, filtered, concentrated in vacuo, washed with hexanes, and filtered to afford the title compound (0.046 g, 90%) as a yellow solid. ¹H NMR (400 MHz, DMSO-d₆) δ ppm 16.1 (s, 1H), 12.9 (br. s., 1H), 12.3 (t, J=5.6 Hz, 1H), 9.24 (d, J=2.3 Hz, 1H), 8.74 (d, J=2.3 Hz, 1H), 8.21 (s, 1H), 7.81 (d, J=8.3 Hz, 2H), 7.61 (d, J=8.3 Hz, 2H), 7.59 (d, J=8.3 Hz, 2H), 7.52 (d, J=8.3 Hz, 2H), 4.28 (d, J=5.6 Hz, 2H), 1.35 (s, 9H), 1.35 (s, 9H). MS (ES+) m/e 511 [M+H]⁺.

### Example 9

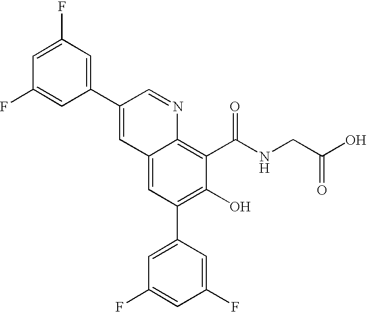

### N-{[3,6-bis(3,5-difluorophenyl)-7-hydroxy-8-quinolinyl]carbonyl}glycine

### 9a) Ethyl N-{[3,6-bis(3,5-difluorophenyl)-7-hydroxy-8-quinolinyl]carbonyl}glycinate

A solution of the compound from Example 7b) (0.050 g, 0.116 mmol), (3,5-difluorophenyl)boronic acid (0.018 g, 0.116 mmol), potassium carbonate (0.048 g, 0.347 mmol), and tetrakis(triphenylphosphine)palladium(0) (0.004 g, 0.0035 mmol) in 1,4-dioxane (1.5 mL) and water (0.5 mL) was heated to 100° C. for 20 min in a Biotage Initiator® microwave synthesizer. Upon cooling, the reaction mixture was concentrated in vacuo and purified via flash column chromatography (0-30% ethyl acetate in hexanes) to afford the title compound (0.045 g, 78%) as a white solid. ¹H NMR (400 MHz, DMSO-d₆) δ ppm 16.2 (s, 1H), 12.3 (t, J=5.8 Hz, 1H), 9.34 (d, J=2.3 Hz, 1H), 8.92 (d, J=2.3 Hz, 1H), 8.31 (s, 1H), 7.71 (dd, J=9.0, 2.4 Hz, 2H), 7.44 (dd, J=8.7, 2.4 Hz, 2H), 7.33-7.41 (m, 2H), 4.38 (d, J=5.8 Hz, 2H), 4.20 (q, J=7.1 Hz, 2H), 1.25 (t, J=7.1 Hz, 3H). MS (ES+) m/e 499 [M+H]⁺.

### 9b) N-{[3,6-bis(3,5-difluorophenyl)-7-hydroxy-8-quinolinyl]carbonyl}glycine

To a solution of the compound from Example 9a) (0.045 g, 0.090 mmol) in tetrahydrofuran (1.0 mL) and methanol (1.0 mL) was added 1N aqueous sodium hydroxide (0.500 mL, 0.500 mmol). After stirring 15 min. at ambient temperature, the reaction was quenched with 1N aqueous hydrochloric acid, diluted with brine, and extracted twice with ethyl acetate. The combined organic layers were dried over MgSO₄, filtered, concentrated in vacuo, washed with hexanes, and filtered to afford the title compound (0.035 g, 82%) as a pale yellow solid. ¹H NMR (400 MHz, DMSO-d₆) δ ppm 16.3 (s, 1H), 13.0 (br. s., 1H), 12.2 (t, J=5.3 Hz, 1H), 9.31 (d, J=2.3 Hz, 1H), 8.87 (d, J=2.3 Hz, 1H), 8.27 (s, 1H), 7.69 (d, J=6.8 Hz, 2H), 7.42 (d, J=6.6 Hz, 2H), 7.35 (t, J=9.2 Hz, 2H), 4.29 (d, J=5.3 Hz, 2H). MS (ES+) m/e 471 [M+H]⁺.

### Biological Background:

The following references set out information about the target enzymes, HIF prolyl hydroxylases, and methods and materials for measuring inhibition of same by small molecules.
M. Hirsilä, P. Koivunen, V. Günzler, K. I. Kivirikko, and J. Myllyharju “Characterization of the Human Prolyl 4-Hydroxylases That Modify the Hypoxia-inducible Factor” J. Biol. Chem., 2003, 278, 30772-30780.
C. Willam, L. G. Nicholls, P. J. Ratcliffe, C. W. Pugh, P. H. Maxwell “The prolyl hydroxylase enzymes that act as oxygen sensors regulating destruction of hypoxia-inducible factor α” Advan. Enzyme Regul., 2004, 44, 75-92
M. S. Wiesener, J. S. Jürgensen, C. Rosenberger, C. K. Scholze, J. H. Hörstrup, C. Warnecke, S. Mandriota, I. Bechmann, U. A. Frei, C. W. Pugh, P. J. Ratcliffe, S. Bachmann, P. H. Maxwell, and K.-U. Eckardt “Widespread hypoxia-inducible expression of HIF-2α in distinct cell populations of different organs” FASEB J., 2003, 17, 271-273.
S. J. Klaus, C. J. Molineaux, T. B. Neff, V. Guenzler-Pukall, I. Lansetmo Parobok, T. W. Seeley, R. C. Stephenson “Use of hypoxia-inducible factor α (HIFα) stabilizers for enhancing erythropoiesis” PCT Int. Appl. (2004), WO 2004108121 A1
C. Warnecke, Z. Zaborowska, J. Kurreck, V. A. Erdmann, U. Frei, M. Wiesener, and K.-U. Eckardt “Differentiating the functional role of hypoxia-inducible factor (HIF)-1α and HIF-2α (EPAS-1) by the use of RNA interference: erythropoietin is a HIF-2α target gene in Hep3B and Kelly cells” FASEB J., 2004, 18, 1462-1464.
   For the expression of EGLN3 see:
R. K. Bruick and S. L. McKnight “A Conserved Family of Prolyl-4-Hydroxylases That Modify HIF” Science, 2001, 294, 1337-1340.
   For the expression of HIF2α-CODD see:
a) P. Jaakkola, D. R. Mole, Y.-M. Tian, M. I. Wilson, J. Gielbert, S. J. Gaskell, A. von Kriegsheim, H. F. Hebestreit, M. Mukherji, C. J. Schofield, P. H. Maxwell, C. W. Pugh, P, J. Ratcliffe “Targeting of HIF-α to the von Hippel-Lindau Ubiquitylation Complex by O₂-Regulated Prolyl Hydroxylation” Science, 2001, 292, 468-472.
b) M. Ivan, K. Kondo, H. Yang, W. Kim, J. Valiando, M. Ohh, A. Salic, J. M. Asara, W. S. Lane, W. G. Kaelin Jr. “HIFα Targeted for VHL-Mediated Destruction by Proline Hydroxylation: Implications for O₂Sensing” Science, 2001, 292, 464-468.
   For the expression of VHL, elongin b and elongin c see:
A. Pause, S. Lee, R. A. Worrell, D. Y. T. Chen, W. H. Burgess, W. M. Linehan, R. D. Klausner “The von Hippel-Lindau tumor-suppressor gene product forms a stable complex with human CUL-2, a member of the Cdc53 family of proteins” Proc. Natl. Acad. Sci. USA, 1997, 94, 2156-2161.

### Biological Assay(s)

### EGLN3 Assay

### Materials:

His-MBP-EGLN3 (6HisMBPAttB1EGLN3(1-239)) was expressed in E. Coli and purified from an amylase affinity column. Biotin-VBC [6HisSumoCysVHL(2-213), 6HisSumoElonginB(1-118), and 6HisSumoElonginC(1-112)] and His-GB1-HIF2α-CODD (6HisGB1tevHIF2A(467-572)) were expressed from E. Coli.

### Method:

Cy5-labelled HIF2α CODD, and a biotin-labeled VBC complex were used to determine EGLN3 inhibition. EGLN3 hydroxylation of the Cy5CODD substrate results in its recognition by the biotin-VBC. Addition of a Europium/streptavidin (Eu/SA) chelate results in proximity of Eu to Cy5 in the product, allowing for detection by energy transfer. A ratio of Cy5 to Eu emission (LANCE Ratio) is the ultimate readout, as this normalized parameter has significantly less variance than the Cy5 emission alone.

Then 50 nL of inhibitors in DMSO (or DMSO controls) were stamped into a 384-well low volume Corning NBS plate, followed by addition of 2.5 μL of enzyme [50 mL buffer (50 mM HEPES/50 mM KCl)+1 mL of a 10 mg/mL BSA in buffer+6.25 μL of a 10 mg/mL FeCl₂ solution in water+100 μL of a 200 mM solution of ascorbic acid in water+15.63 μL EGLN3] or control [50 mL buffer+1 mL of a 10 mg/mL BSA in buffer+6.25 μL of a 10 mg/mL FeCl₂ solution in water+100 μL of a 200 mM solution of ascorbic acid in water]. Following a 3 minutes incubation, 2.5 μL of substrate [50 mL Buffer+68.6 μL biotin-VBC+70.4 μL Eu (at 710 μg/mL stock)+91.6 μL Cy5CODD+50 μL of a 20 mM solution of 2-oxoglutaric acid in water+0.3 mM CHAPS] was added and incubated for 30 minutes. The plate was loaded into a PerkinElmer Viewlux for imaging. For dose response experiments, normalized data were fit by ABASE/XC50 using the equation y=a+(b−a)/(1+(10̂x/10̂c) ̂d), where a is the minimum % activity, b is the maximum % activity, c is the pIC₅₀, and d is the Hill slope.

The IC₅₀ for exemplified compounds in the EGLN3 assay ranged from approximately 1-100 nanomolar. This range represents the data accumulated as of the time of the filing of this initial application. Later testing may show variations in IC₅₀ data due to variations in reagents, conditions and variations in the method(s) used from those given herein above. So this range is to be viewed as illustrative, and not a absolute set of numbers.

### Measure Epo Protein Produced by Hep3B Cell Line Using ELISA Method.

Hep3B cells obtained from the American Type Culture Collection (ATCC) are seeded at 2×10̂4 cells/well in Dulbecco's Modified Eagle Medium (DMEM)+10% FBS in 96-well plates. Cells are incubated at 37 degC/5% CO2/90% humidity (standard cell culture incubation conditions). After overnight adherence, medium is removed and replaced with DMEM without serum containing test compound or DMSO negative control. Following 48 hours incubation, cell culture medium is collected and assayed by ELISA to quantitate Epo protein.

The EC₅₀ for exemplar compounds in the Hep3B ELISA assay ranged from approximately 1-20 micromolar using the reagents and under the conditions outlined herein above. This range represents the data accumulated as of the time of the filing of this initial application. Later testing may show variations in EC₅₀ data due to variations in reagents, conditions and variations in the method(s) used from those given herein above. So this range is to be viewed as illustrative, and not a absolute set of numbers.

These compound are believed to be useful in therapy as defined above and to not have unacceptable or untoward effects when used in compliance with a permitted therapeutic regime.

The foregoing examples and assay have been set forth to illustrate the invention, not limit it. What is reserved to the inventors is to be determined by reference to the claims.

1. A a compound of formula (I): 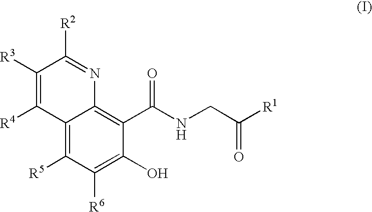
wherein:
R¹ is —NR⁷R⁸ or —OR⁹;
R², R³, R⁴, R⁵, and R⁶ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CONR¹⁰R¹¹, —N(R¹⁰)C(O)R¹², —N(R¹⁰)C(O)OR¹², —OC(O)NR¹⁰R¹¹, —N(R¹⁰)C(O)N¹⁰R¹¹, —P(O)(OR¹²)₂, —SO₂NR¹⁰R¹¹, —N(R¹⁰)SO₂R¹², C₁-C₁₀ alkyl, C₁-C₁₀ alkenyl, C₁-C₁₀ alkynyl, C₃-C₈ cycloalkyl, C₃-C₈ heterocycloalkyl, C₅-C₈ cycloalkenyl, aryl, and heteroaryl;
R⁷ and R⁸ are each independently selected from the group consisting of hydrogen, C₁-C₆ alkyl, C₂-C₆ alkenyl, C₂-C₆ alkynyl, C₃-C₆ cycloalkyl, C₃-C₆ heterocycloalkyl, aryl, and heteroaryl;
R⁹ is hydrogen, or a cation, or C₁-C₄ alkyl;
R¹⁰ and R¹¹ are each independently selected from the group consisting of hydrogen, C₁-C₁₀ alkyl, C₃-C₈ cycloalkyl, alkyl-C₃-C₈ cycloalkyl, C₃-C₈ heterocycloalkyl, C₁-C₁₀ alkyl-C₃-C₈ heterocycloalkyl, aryl, C₁-C₁₀ alkyl-aryl, heteroaryl, C₁-C₁₀ alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;
each R¹² is independently selected from the group consisting of hydrogen, C₁-C₁₀ alkyl, C₂-C₁₀ alkenyl, C₂-C₁₀ alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈ heterocycloalkyl, aryl, C₁-C₁₀ alkyl-aryl, heteroaryl, and C₁-C₁₀ alkyl-heteroaryl;
any carbon or heteroatom of R², R³, R⁴, R⁵, R⁶, R⁷, R⁸, R⁹, R¹⁰, R¹¹, or R¹² is unsubstituted or, where possible, is substituted with one or more substituents independently selected from C₁-C₆ alkyl, aryl, heteroaryl, halogen, —OR¹², —NR¹⁰R¹¹ cyano, nitro, —C(O)R¹², —C(O)OR¹², —SR¹², —S(O)R¹², —S(O)₂R¹², —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₁-C₁₀ alkenyl, C₁-C₁₀ alkynyl, C₃-C₈ cycloalkyl, C₃-C₈ heterocycloalkyl, C₅-C₈ cycloalkenyl, aryl or heteroaryl, wherein R¹⁰, R¹¹, and R¹² are the same as defined above; or a pharmaceutically acceptable salt or solvate thereof.

2. A compound according to claim 1 wherein:
R¹ is —OR⁹;
R², R³, R⁴, R⁵, R⁶ are each independently selected from the group consisting of hydrogen, cyano, halogen, —OR¹², —NR¹⁰R¹¹, —CONR¹⁰R¹¹, C₁-C₆ alkyl, C₃-C₆ cycloalkyl, C₃-C₆ heterocycloalkyl, aryl, and heteroaryl;
R⁹ is hydrogen, or a cation;
R¹⁰ and R¹¹ are each independently selected from the group consisting of hydrogen, C₁-C₆ alkyl, C₃-C₆ cycloalkyl, C₃-C₆ heterocycloalkyl, aryl, heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;
each R¹² is independently selected from the group consisting of hydrogen, C₁-C₆ alkyl, C₂-C₆ alkenyl, C₂-C₆ alkynyl, —CO(C₁-C₄ alkyl), —CO(aryl), —CO(heteroaryl), —CO(C₃-C₆ cycloalkyl), —CO(C₃-C₆ heterocycloalkyl), C₃-C₆ cycloalkyl, C₃-C₆ heterocycloalkyl, aryl, and heteroaryl;
any carbon or heteroatom of R², R³, R⁴, R⁵, R⁶, R⁹, R¹⁰, R¹¹, or R¹² is unsubstituted or, where possible, is substituted with one or more substituents independently selected from C₁-C₆ alkyl, aryl, heteroaryl, halogen, —OR¹², —NR¹⁶R¹¹, cyano, —C(O)R¹², —C(O)OR¹², —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰, R¹¹, and R¹² are the same as defined above;
or a pharmaceutically acceptable salt or solvate thereof.

3. A compound according to claim 1 wherein:
R¹ is —OR⁹;
R², R⁴, and R⁵ are each hydrogen;
R³ and R⁶ are each independently selected from the group consisting of hydrogen, cyano, halogen, —OR¹², —NR¹⁰R¹¹, —CONR¹⁰R¹¹, C₁-C₆ alkyl, C₃-C₆ cycloalkyl, C₃-C₆ heterocycloalkyl, aryl, and heteroaryl;
R⁹ is hydrogen, or a cation;
R¹⁰ and R¹¹ are each independently selected from the group consisting of hydrogen, C₁-C₆ alkyl, C₃-C₆ cycloalkyl, C₃-C₆ heterocycloalkyl, aryl, and heteroaryl; or
R¹⁶ and R¹¹ taken together with the nitrogen to which they are attached form a 5- or 6- or 7-membered saturated ring optionally containing one other heteroatom which is oxygen, nitrogen or sulphur;
each R¹² is independently selected from the group consisting of hydrogen, C₁-C₆ alkyl, C₃-C₆ cycloalkyl, C₃-C₆ heterocycloalkyl, aryl, and heteroaryl;
any carbon or heteroatom of R³, R⁶, R⁹, R¹⁰, R¹¹, or R¹² is unsubstituted or, where possible, is substituted with one or more substituents independently selected from C₁-C₆ alkyl, aryl, heteroaryl, halogen, —OR¹², —NR¹⁰R¹¹, cyano, —C(O)R¹², —C(O)OR¹², —CONR¹⁰R¹¹, —(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₂-C₆ alkenyl, C₂-C₆ alkynyl, C₃-C₆ cycloalkyl, C₃-C₆ heterocycloalkyl, C₅-C₈ cycloalkenyl, aryl, or heteroaryl, wherein R¹⁰, R¹¹, and R¹² are the same as defined above;
or a pharmaceutically acceptable salt or solvate thereof.

4. A compound according to claim 1 which is:
N-[(7-hydroxy-8-quinolinyl)carbonyl]glycine;
N-[(7-hydroxy-3-phenyl-8-quinolinyl)carbonyl]glycine;
N-[(3-bromo-7-hydroxy-8-quinolinyl)carbonyl]glycine;
N-({3-[4-(1-dimethylethyl)phenyl]-7-hydroxy-8-quinolinyl}carbonyl)glycine;
N-({7-hydroxy-3-[4-(trifluoromethyl)phenyl]-8-quinolinyl}carbonyl)glycine;
N-[(3,6-dibromo-7-hydroxy-8-quinolinyl)carbonyl]glycine;
N-[(7-hydroxy-3,6-diphenyl-8-quinolinyl)carbonyl]glycine;
N-({3,6-bis[4-(1,1-dimethylethyl)phenyl]-7-hydroxy-8-quinolinyl}carbonyl)glycine; and
N-{[3,6-bis(3,5-difluorophenyl)-7-hydroxy-8-quinolinyl]carbonyl}glycine;
or a pharmaceutically acceptable salt or solvate thereof.

5. A method for treating anemia in a mammal, which method comprises administering an effective amount of a compound of formula (I) or a salt or solvate thereof according to claim 1 to a mammalian suffering from anemia which can be treated by inhibiting HIF prolyl hydroxylases.

6. A pharmaceutical composition comprising a compound of formula (I) or a salt, solvate, according to claim 1 and one or more of pharmaceutically acceptable carriers, diluents and excipients.

7. A process for preparing a compound of formula (I) 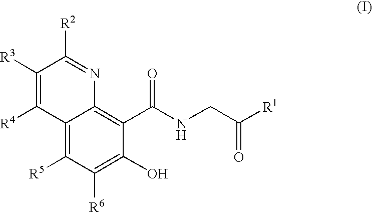
wherein R¹, R², R³, R⁴, R⁵, and R⁶ are the same as defined above for formula (I), the process
comprising treating a compound of formula A: 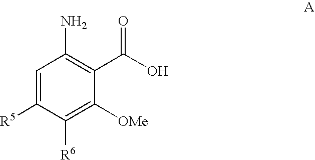
wherein R⁵ and R⁶ are the same as for those groups in formula (I), with an appropriately substituted α,β-unsaturated carbonyl compound, such as acrolein, 2-phenylpropenal, or 2-bromoacrolein, in an appropriate solvent, such as acetic acid or 1,4-dioxane, with or without the addition of bromine, with heating under either conventional thermal conditions or by microwave irradiation, to form a compound of formula B: 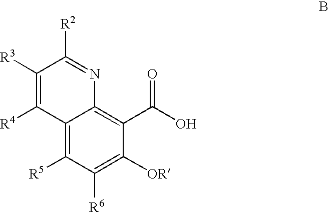
wherein R², R³, R⁴, R⁵, and R⁶ are the same as for those groups in formula (I) and R′ is H or Me, which is coupled with an appropriate glycine ester, such as glycine ethyl ester hydrochloride, and an appropriate base, such as triethylamine, and an appropriate coupling reagent, such as HATU, in an appropriate solvent, such as N,N-dimethylformamide, followed by ester hydrolysis with an appropriate base, such as sodium hydroxide, in an appropriate solvent, such as tetrahydrofuran and/or methanol, or when necessary, ether cleavage/ester hydrolysis with an appropriate reagent, such as boron tribromide, in an appropriate solvent, such as dichloromethane, to form a compound of formula (I) where R¹ is —OH.
